(19) United States
(12) Patent Application Publication
(10) Pub. No.: US 2026/0263361 A1
(43) Pub. Date: Sep. 10, 2026

(54) CANNABIDIOL AQUEOUS SOLUTION COMPOSITION AND CANNABIDIOL-CONTAINING PHARMACEUTICAL PREPARATION COMPRISING SAME

(71) Applicant: CTC BIO, INC., Gyeonggi-do (KR)

(72) Inventor: Su-Jun PARK, Gyeonggi-do (KR)

(21) Appl. No.: 19/166,206

(22) PCT Filed: Mar. 15, 2024
(86) PCT No.: PCT/KR2024/003314

§ 371 Date: Sep. 17, 2025

(30) Foreign Application Priority Data
Mar. 17, 2023 (KR) 10-2023-0035440
May 31, 2023 (KR) 10-2023-0069795
May 31, 2023 (KR) 10-2023-0069796
May 31, 2023 (KR) 10-2023-0069797

(51) Int. Cl. A61K 9/08; A61K 9/00; A61K 31/00; A61K 47/10; A61K 47/22; A61K 47/26; A61K 47/34; A61K 47/36

(52) U.S. Cl. CPC A61K 9/08; A61K 9/006; A61K 31/658; A61K 47/10; A61K 47/22; A61K 47/26; A61K 47/34; A61K 47/36

(57) ABSTRACT

The present invention provides a cannabidiol aqueous solution composition containing water as a solvent, comprising (a) cannabidiol and (b) additives comprising an ester of tocopherol, polysorbate and poloxamer. The aqueous solution composition is advantageous for the solubilization of cannabidiol and can improve the solubility of cannabidiol by using a small amount of a solubilizing agent. An oral disintegrating film prepared including the composition has excellent over-time stability of cannabidiol.

### TECHNICAL FIELD

This application is based on and claims priority from Korean Patent Application No. 10-2023-0069795, filed on May 31, 2023, Korean Patent Application No. 10-2023-0069796 filed on May 31, 2023, Korean Patent Application No. 10-2023-0069797 filed on May 31, 2023, and Korean Patent Application No. 10-2023-0035440 filed on Mar. 17, 2023, with the Korean Intellectual Property Office, the disclosure of which is incorporated herein in its entirety by reference. The present invention relates to a cannabidiol aqueous solution composition and a pharmaceutical preparation comprising the same, and more specifically, to a cannabidiol aqueous solution composition with improved solubility and stability of cannabidiol and a pharmaceutical preparation comprising the said aqueous solution composition.

### BACKGROUND ART

Cannabidiol (CBD) is a cannabinoid that is chemically represented as 2-[(1R,6R)-3-methyl-6-(1-methylethenyl)-2-cyclohexen-1-yl]-5-pentyl-1,3-benzenediol. Its empirical formula is C21H30O2, and its molecular weight is 314.46. CBD is a cannabinoid that naturally occurs in the plant Cannabis sativa L. Pure CBD generally exists as oil or as an isolate, and among these, the isolate is a white to pale yellow crystalline solid that is insoluble in water and soluble in organic solvents.

CBD (cannabidiol) is a phytocannabinoid discovered in 1940. Cannabinoids are natural and synthetic compounds that are constituents of the Cannabis plant or that are structurally or pharmacologically related to endogenous agonists of the cannabinoid receptors CB1 or CB2 (endocannabinoids). Cannabidiol (CBD), which is one of the cannabinoids, is known as a substance exhibiting excellent therapeutic efficacy and effects. It has been reported that CBD can be used for the treatment of asthma, glaucoma, various cancers, Parkinson's disease, seizures, dementia, arthritis, obesity, and anxiety disorders. In addition, it is also known to have effects such as antibacterial effects and relaxation of tension.

Among the cannabinoids contained in cannabis, the substance having the greatest psychotropic effect is delta-9-tetrahydrocannabinol (THC). A purified cannabidiol isolate, from which THC, which has significant harm to the human body, has been removed, has generally been provided as CBD oil.

The CBD isolate is a purified form of cannabidiol (CBD) in which other compounds have been removed from hemp-derived cannabidiol (cannabidiol). The CBD isolate is in the form of a powder and has remarkably poor solubility in water, making it difficult to provide as an aqueous solution, and there is a problem of limitation of dosage forms due to difficulty in blending with water-soluble polymer films. Due to this, cannabidiol is highly hydrophobic and does not dissolve in water, and in this regard, there are many patents that attempt to increase the bioavailability of CBD by using compositions that use some organic solvent.

In addition, CBD has been unstable under storage conditions of pH, temperature, or light, making formulation difficult. CBD has poor long-term stability and tends to reduce the content of CBD in the formulation over time, which has been confirmed to cause a problem of reduced drug efficacy when manufactured as a pharmaceutical. The CBD isolate is basically dispersed in an oil phase, and when mixed in oil, one of the biggest factors of stability reduction can be cited as rancidity due to oxidation. The inventors of the present invention studied measures that can reduce the influence on nonbonding electron pairs in oxygen molecules in order to suppress rancidity due to oxidation, and studied the structure of cannabidiol, and, without mixing in an oil phase, arrived at completing a CBD aqueous solution and a CBD-containing preparation that is solubilized in water and has improved long-term stability.

### DISCLOSURE

### Technical Problem

The present disclosure is designed to solve the above problems, and is therefore directed to providing a CBD aqueous solution composition that exhibits improved water solubility while stably containing CBD, as well as a pharmaceutical preparation comprising the same. Without the use of organic solvents to dissolve CBD, a stable colloidal state in 100% water can be achieved, and the disintegration rate is remarkably improved. In addition, a formulation capable of dissolving in water can be provided.

The present disclosure is also directed to providing a CBD aqueous solution composition which stably contains more than 100 mg of CBD (particularly powdered CBD) per 1 mL of water and passes through a membrane filter with a pore size of 0.45 μm or less at a rate of 90% or more.

The present disclosure is further designed to solve the above problems and is therefore directed to providing a novel CBD aqueous solution, a CBD-containing preparation, or a method for preparing the same, capable of stably containing CBD.

The present disclosure is also directed to providing an oral disintegrating film containing CBD with excellent long-term stability, thereby addressing the instability of CBD, which has been a problem in conventional CBD-containing preparations.

### Technical Solution

In one aspect of the present disclosure, there is provided a cannabidiol (CBD) aqueous solution composition having excellent solubility. Particularly, there is provided a cannabidiol aqueous solution composition comprising a purified cannabidiol isolate having a purity of 99% or higher. Additionally, in one aspect of the present disclosure, there is provided a cannabidiol aqueous solution or a cannabidiol preparation comprising the same, in which the stability of cannabidiol in the formulation is improved.

In one aspect of the present disclosure, there is provided a cannabidiol aqueous solution composition comprising water as a solvent,
the composition comprising: (a) cannabidiol; and
(b) an additive comprising an ester of tocopherol, a polysorbate, and a poloxamer.

Preferably, the solvent substantially does not comprise any organic solvent other than water. As used herein, “substantially does not comprise” means comprising less than 3 wt %, less than 2.5 wt %, less than 2 wt %, less than 1 wt %, less than 0.5 wt %, or less than 0.1 wt % based on the total weight of the solvent, or comprising none at all. The organic solvent may be selected from among polar solvents and nonpolar solvents, and may include commonly used hydrocarbons, alcohols, ethers, ketones, butanol, and amines, and may include, for example, acetic acid, methanol, ethanol, propanol, butanol, acetone, and chloroform. The additive comprises an ester of tocopherol, a polysorbate, and a poloxamer. In one aspect of the present disclosure, there is provided a cannabidiol aqueous solution composition in which not only solubility but also stability is improved. As used herein, cannabidiol preferably comprises a cannabidiol isolate, and the cannabidiol isolate (or cannabidiol purified material) refers to pure cannabidiol obtained from plants called hemp or cannabis, from which other compounds have been removed and which contains less than 1% of impurities.

In one aspect of the present disclosure, the cannabidiol for which solubility can be improved includes powdered cannabidiol, is preferably in powdered form, may comprise high-purity crystalline isolated cannabidiol, and more preferably comprises high-purity crystalline isolated cannabidiol having an impurity content of less than 1%. That is, it may comprise CBD powder having a purity of 99%. As used herein, a CBD isolate may be understood as CBD in powdered form.

In one aspect of the present disclosure, the inventors have confirmed that, in order to prepare an aqueous solution of cannabidiol, particularly crystalline isolated cannabidiol, it is preferable to include, as additives, an ester of tocopherol, a polysorbate, and a poloxamer. A cannabidiol aqueous solution containing the additives facilitates solubilization of a cannabidiol isolate, making it easy to prepare an aqueous solution of a CBD isolate, and exhibits excellent long-term stability of CBD in film preparations containing CBD.

In another aspect of the present disclosure, the aqueous solution composition may further comprise, as the additive (b), alpha-tocopherol (α-tocopherol). By further comprising alpha-tocopherol in the additive (b), the long-term stability of CBD in an oral dissolving film prepared from the aqueous solution composition can be improved.

The additive (b) is described in greater detail.

Preferably, the additive (b) may be a solubilizer for solubilizing the cannabidiol in water.

In one embodiment of the present disclosure, the ester of tocopherol is an ester derivative of tocopherol, and the ester of tocopherol may comprise one or more selected from the group consisting of tocopheryl acetate (acetic acid ester of tocopherol), tocopheryl linoleate (linoleic acid ester of tocopherol), tocopheryl linoleate/oleate (a mixture of the linoleic acid ester and oleic acid ester of tocopherol), tocopheryl nicotinate (nicotinic acid ester of tocopherol), and tocopheryl succinate (succinic acid ester of tocopherol), and preferably may comprise one or more of tocopheryl acetate and tocopheryl linoleate, and more preferably may comprise tocopheryl acetate. When used together with a polysorbate and a poloxamer, the ester of tocopherol can improve the solubility of cannabidiol in water, and in particular, tocopheryl acetate can be advantageously used for the solubility and/or stability of cannabidiol.

The composition comprises a polysorbate, and may comprise one or more selected from the group consisting of polysorbate 20, polysorbate 40, polysorbate 60, and polysorbate 80, preferably one or more selected from the group consisting of polysorbate 20 and polysorbate 40, and most preferably may comprise polysorbate 20. Polysorbate 20 has excellent compatibility with other additives, provides excellent solubilization effects, and is advantageous for improving the long-term stability of CBD.

The composition comprises a poloxamer, and the poloxamer comprises one or more selected from the group consisting of poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407, and preferably comprises one or more selected from the group consisting of poloxamer 181, poloxamer 182, poloxamer 183, poloxamer 184, poloxamer 185, and poloxamer 188, and more preferably may comprise poloxamer 188. The composition, particularly when poloxamer 188 and polysorbate 20 are used together, can improve the solubility or dispersibility of cannabidiol, allow cannabidiol to exist in a solution state, and reduce or diminish phenomena of recrystallization or precipitation.

As used herein, the terms “improved solubility” or “solubilized” can be understood to mean that a substance that is not easily soluble in water (particularly the CBD isolate of the present disclosure) is uniformly dispersed in water or maintained in a stable solution state. For example, it may exist in the form of an emulsion.

In one aspect of the present disclosure, the term “improved stability,” as used herein, means that the content of cannabidiol in the aqueous solution or preparation is not reduced or is hardly reduced compared to the initial content at the time of manufacture. Specifically, it may refer to a change in CBD content within ±10 wt % relative to the content at the time of initial manufacture, more preferably within ±8 wt %, more preferably within ±7 wt %, more preferably within ±6 wt %, more preferably within ±5 wt %, even more preferably within ±4 wt %, and most preferably within ±3 wt % of the content at the time of initial manufacture.

In one embodiment of the present disclosure, there is provided an aqueous solution composition of cannabidiol, wherein the aqueous solution comprises water as a solvent and provides an aqueous solution composition comprising or consisting of cannabidiol, tocopheryl acetate, a polysorbate, and a poloxamer. Preferably, the aqueous solution composition provides an aqueous solution composition comprising or consisting of a cannabidiol isolate, tocopheryl acetate, polysorbate 20, and poloxamer 188. The aqueous solution composition can improve the solubility of cannabidiol, allow cannabidiol to exist in a solution state, and prevent recrystallization or precipitation. The cannabidiol can be miscible in the aqueous solution comprising water as the solvent and, preferably, can be uniformly dispersed or dissolved. The aqueous solution composition exhibits a markedly reduced change in cannabidiol content even after long-term storage.

According to one embodiment, the aqueous solution composition may form microcapsules as the cannabidiol mixed with the additive forms a small average particle size, thereby improving water solubility and enhancing in vivo absorption. Preferably, the cannabidiol dispersed in the aqueous solution composition may have an average particle size of 0.5 μm or less. In the cannabidiol aqueous solution according to the present disclosure, at least 90% or more, more preferably 92% or more, and still more preferably 95% or more of the total amount of cannabidiol dispersed or dissolved in the composition can pass through a 0.45 μm membrane filter. This can be understood to mean that cannabidiol is uniformly dispersed in the aqueous solution and that no precipitation is formed.

In one embodiment of the present disclosure, the aqueous solution composition may be mixed in a content ratio of the cannabidiol and the additive suitable for homogenizing the cannabidiol. Preferably, the cannabidiol and the additive included in the aqueous solution composition are mixed in a weight ratio of 1:0.1-10, preferably 1:0.5-5, and more preferably 1:0.8-1.5. In one embodiment, the additive may be included in an amount of 4 to 25 wt %, preferably 5 to 23 wt %, and more preferably 5 to 20 wt % based on the total weight of the aqueous solution composition comprising water. Unlike conventional techniques that require large amounts of additives to dissolve an insoluble component such as a CBD isolate, the additive combination of the present disclosure can stably contain a large amount of cannabidiol (for example, 100 mg or more per 1 mL of water) even with a small amount as described above.

In another embodiment of the present disclosure, the additive may comprise an ester of tocopherol, a polysorbate, and a poloxamer to improve solubility, and each of the components used as the additive is mixed at a weight ratio of 1:0.05-3:0.05-3, preferably 1:0.1-2:0.1-2, more preferably 1:0.2-1.5:0.2-1.5, and most preferably is mixed and used at a weight ratio of 1:0.2-1.5:0.2-1.5. Within these content ranges, achieving the objectives of the present disclosure can be advantageous, and the solubility of cannabidiol can be excellent and the precipitation rate can be significantly improved.

In another embodiment of the present disclosure, the aqueous solution composition may further comprise alpha-tocopherol (α-tocopherol) as the additive. That is, alpha-tocopherol (α-tocopherol) may be further included in the combination of additives comprising the ester of tocopherol, the polysorbate, and the poloxamer. Alpha-tocopherol, when mixed with a polysorbate and a poloxamer without an ester of tocopherol, inhibits solubilization of CBD; however, when used together with the ester of tocopherol, it not only exhibits excellent solubilization but can also provide an outstanding effect on the long-term stability of CBD. In particular, the ester of tocopherol preferably comprises tocopheryl acetate. More preferably, the alpha-tocopherol may be included in the aqueous solution composition in the same amount as the ester of tocopherol.

As used herein, unless otherwise stated, the content of the additives included in the cannabidiol aqueous solution composition and the blending ratio of the additive to cannabidiol shall be understood to include cases where alpha-tocopherol is included in the additive (b). The alpha-tocopherol may be included in the CBD aqueous solution but may also be mixed separately from the CBD aqueous solution during the process of manufacturing an orally disintegrating film containing CBD. The order of mixing the alpha-tocopherol is not particularly limited.

In one embodiment of the present disclosure, there is provided a method for preparing a cannabidiol isolate aqueous solution and a method for preparing a CBD-containing orally disintegrating film comprising the aqueous solution composition.

Preferably, one embodiment provides a method for solubilizing a cannabidiol isolate in water. In one embodiment, the method comprises: (S1) preparing a mixture by mixing a cannabidiol isolate and an additive; (S2) adding the mixture prepared in step (S1) to a solvent comprising water; and (S3) stirring the solvent containing the mixture of step (S2). The cannabidiol isolate has an impurity content of less than 1%, and the additive comprises an ester of tocopherol, a polysorbate, and a poloxamer.

According to another embodiment, the additive of step (S1) may further comprise alpha-tocopherol as needed. The additive may comprise both an ester of tocopherol and alpha-tocopherol, and when alpha-tocopherol is included, the content of alpha-tocopherol may be added in the same amount as the ester of tocopherol.

Specifically, the cannabidiol additive may be included in an amount of 4 to 25 wt % based on the total weight of the aqueous solution composition comprising water.

The ester of tocopherol may comprise one or more selected from the group consisting of tocopheryl acetate, tocopheryl linoleate, tocopheryl linoleate/oleate, tocopheryl nicotinate, and tocopheryl succinate.

The polysorbate may comprise one or more selected from the group consisting of polysorbate 20, polysorbate 40, polysorbate 60, and polysorbate 80.

The poloxamer may comprise one or more selected from the group consisting of poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407. Preferably, the additive may comprise cannabidiol, tocopheryl acetate, polysorbate 20, and poloxamer 188.

The cannabidiol and the additive may be mixed at a weight ratio of 1:0.1-10, preferably 1:0.5-5, and more preferably 1:0.8-1.5. Preferably, the mixing ratio of cannabidiol:additive is understood as the mixing ratio of cannabidiol to the mixture of the ester of tocopherol, the polysorbate, and the poloxamer, or as the mixing ratio of cannabidiol to the mixture of alpha-tocopherol, the ester of tocopherol, the polysorbate, and the poloxamer.

The additive may be mixed and used at a weight ratio of tocopherol ester:polysorbate:poloxamer of 1:0.05-3:0.05-3, preferably 1:0.1-2:0.1-2, more preferably 1:0.2-1.5:0.2-1.5, and most preferably 1:0.2-1.5:0.2-1.5.

In step (S3), the stirring may be performed such that the aqueous solution composition containing more than 100 mg of CBD per 1 mL of water passes a membrane filter of 0.45 μm or less in an amount of 90% or more of the total content of the aqueous solution composition.

The composition prepared by the solubilization method of the present disclosure may exhibit no precipitation without change in content for up to 2 months in a 40° C. accelerated test.

Preferably, the aqueous solution composition may have a pH of 5 to 8.5, preferably a pH of 5.5 to 8, more preferably a pH of 6 to 7.5, and more preferably a pH of 6.5 to 7. In the pH range, excellent solubility can be exhibited. Additionally, as needed, the composition may further comprise step (S4) of sterilization. Various methods commonly used in the industry may be used for the sterilization.

The aqueous solution composition may have a water temperature of 20 to 65° C. at 1 atm, and preferably a temperature of 25 to 55° C. In the temperature range, solubility can be excellent, and mixing with the additive is excellent, resulting in an aqueous solution composition that dissolves well in water and is stable.

In step (S1), the mixture may be prepared by mixing at a water temperature of 20 to 65° C. at 1 atm.

In step (S2), the solvent comprises water, and the water may be used without limitation, including hard water, soft water, seawater, brackish water, fresh water, groundwater, and distilled water. The solvent, in addition to water, may use various additional solvents within a limited range in consideration of the compatibility of the additive, so long as the additive does not affect the solubilization of cannabidiol.

In step (S3), stirring may be performed using various stirrers. The stirring may be performed at room temperature (25° C.). Equipment for stirring, such as a continuous stirred-tank reactor, homogenizer, turbine agitator, and paddle mixer, may be used without limitation.

In another embodiment, the additive of step (S1) comprises tocopheryl acetate, polysorbate 20, and poloxamer 188, and may further comprise alpha-tocopherol. In one embodiment, in consideration of solubility and long-term stability, the order of mixing the additives of step (S1) may be such that tocopheryl acetate, polysorbate 20, and poloxamer 188 are first mixed with cannabidiol and then alpha-tocopherol is mixed.

In one embodiment of the present disclosure, there is provided a cannabidiol oral dissolving preparation comprising a water-soluble polymer for film formation and the cannabidiol aqueous solution composition according to one embodiment of the present disclosure. The oral dissolving (and/or disintegrating) preparation is not particularly limited in form, and preferably any dosage form may be used without limitation so long as at least 80% of the entire film is dissolved, dispersed, or disintegrated within 2 minutes, preferably within 1 minute 30 seconds, and even more preferably within 1 minute after oral administration. The oral dissolving (and/or disintegrating) preparation can be used interchangeably with the terms film, strip, and orally disintegrating film, and is understood to refer to a dosage form that is adhered in the oral cavity, such as on the tongue, oral mucosa, or sublingually, and dissolved for administration.

The oral dissolving preparation may further comprise a thickener, a flavoring agent, and/or a sweetener. The thickeners, flavoring agents, and sweeteners commonly included in the industry may all be included without limitation; however, the thickener may comprise one or more selected from the group consisting of xanthan gum, guar gum, and gum arabic, and preferably, for the purposes of the present disclosure, may comprise xanthan gum, gum arabic, or a mixture thereof. The thickener may be included in an amount of 3 to 10 wt % or 4 to 8 wt % based on the total weight of the dried film to provide viscosity and dryness suitable for preparing the water-soluble polymer into a film. In addition, the oral dissolving film of the present disclosure may further comprise a sweetener to mask the bitterness of cannabidiol in the mouth, and as the sweetener, one or more selected from the group consisting of sucralose, neohesperidin, aspartame, stevioside, sorbitol, and mannitol may be used, and more preferably two or more sweeteners are used to mask the bitterness of cannabidiol. The total weight of the sweetener included in the entire dried film may be 1 to 10 wt %, and preferably 3 to 9 wt %.

In one embodiment of the present disclosure, the water-soluble polymer is used as a base capable of forming a film, and preferably polyvinyl alcohol may be used as a film-forming base. Preferably, considering film formability, dispersibility of cannabidiol, solubility, and processability (viscosity of the blend), among polyvinyl alcohols having various molecular weights and degrees of saponification, a polymer having a weight-average molecular weight of 1,000 to 100,000 is preferred, more preferably 10,000 to 50,000, even more preferably 15,000 to 60,000, and most preferably 20,000 to 30,000 may be used.

In view of the solubility, dispersibility, and formability of cannabidiol, the film may be prepared by mixing the cannabidiol and the water-soluble polymer at a weight ratio of 1:0.1-5, preferably 1:0.5-3, more preferably 1:0.8-1.5, and still more preferably 1:0.9-1.2. Within these content ranges, achieving the objectives of the present disclosure can be advantageous.

The present disclosure may contain a large amount of cannabidiol in the cannabidiol oral dissolving film. Specifically, when using the additives of the present disclosure, the cannabidiol may be included in an amount of 10 to 60 wt %, preferably 13 to 50 wt %, and more preferably 15 to 30 wt % based on the total weight of the film.

The aqueous solution composition of the present disclosure exhibits a remarkably low CBD precipitation rate, excellent solubility and stability, and can stably contain more than 100 mg of CBD per 1 mL of water, while allowing 90% or more of the total content of the aqueous solution composition to pass through a membrane filter of 0.45 μm or less.

The formulation of the present disclosure comprising CBD may have a change in CBD content within ±10 wt % compared to the initial content at the time of manufacture when stored for 3-4 days at a temperature of 60° C. or higher, more preferably 75-85° C.

The oral disintegrating preparation (or film) may comprise alpha-tocopherol (α-tocopherol).

### Advantageous Effects

In one aspect of the present disclosure, a large amount of cannabidiol can be dissolved in water. Even with a small amount of additive, a large quantity of cannabidiol can be dissolved in water. Particularly, when manufacturing a film formulation, the present disclosure has the significant advantage of being able to improve the solubility and/or stability of CBD with a minimal amount of additive, since it is not possible to use a large amount of additive relative to the base component.

Furthermore, the present disclosure does not require the use of any organic solvent to dissolve CBD in water, making it highly advantageous in terms of manufacturing, handling, and safety.

The present disclosure can also improve the stability of CBD.

### DESCRIPTION OF DRAWINGS

The accompanying drawings illustrate a preferred embodiment of the present disclosure and together with the foregoing disclosure, serve to provide further understanding of the technical features of the present disclosure, and thus, the present disclosure is not construed as being limited to the drawing.

FIG. 1 shows the state in which the CBD isolate is not dissolved. In FIG. 1, from the left, each image shows an aqueous solution containing CBD, where glycerin, lecithin, polyethylene glycol 400, simethicone emulsion 30%, and polysorbate 80 are each used as a solubilizer by being mixed with corn oil in the aqueous solution composition. It was confirmed that the CBD was not dissolved and appeared as precipitate or crystals.

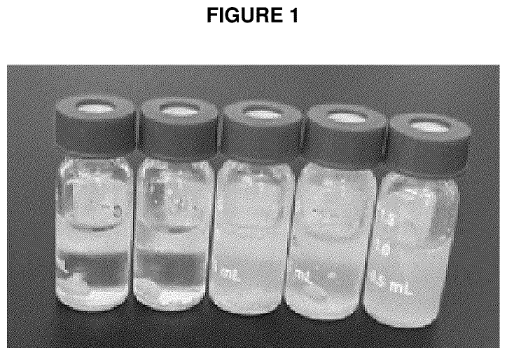

FIG. 2 shows the final mixture state of the CBD aqueous solution composition having the composition of Table 5. The aqueous solution composition containing CBD isolate exhibited a stably emulsified appearance.

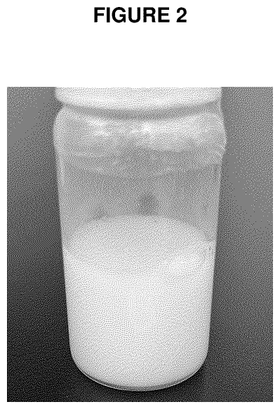

FIG. 3 shows the results of checking the degree of CBD precipitation in Comparative Example 1 (left) and Example 1 (right). When tocopheryl acetate was added (right), the CBD emulsion was stably formed, whereas when tocopherol was added (left), the CBD was precipitated in a crystalline form.

FIG. 4 shows the results of comparing the stability of CBD in the CBD-containing composition.

### MODE FOR DISCLOSURE

Hereinafter, embodiments and the like will be described in detail to aid the understanding of the present disclosure. However, the embodiments according to the present disclosure may be modified in various other forms, and the scope of the present disclosure should not be construed as being limited to the following embodiments. The embodiments of the present disclosure are provided to more fully illustrate the present disclosure to those of ordinary skill in the art. Unless otherwise specified, the “%” used in this specification is to be understood as weight percent.

### [Preliminary Experiment for Preparation of CBD Aqueous Solution Composition]

A screening test was conducted to evaluate the individual and/or combined effects of each component for dissolving powdered CBD isolate in water.

The experiment was performed as follows.

CBD and each component were mixed at a 1:1 ratio, and the state of the mixture was observed visually. Specifically, after adding water to the mixed solution, the state was observed.

The symbol “O” indicates no precipitation after the addition of water; “Δ” indicates that some solids remain before water addition or precipitation occurs after water addition; and “X” indicates that the component does not dissolve in the absence of added water.

TABLE 1

| | | CBD isolate |
|---|---|---|
| Number | List of Materials | solubilization |
| 1 | Corn oil | ◯ |
| 2 | Glycerin | X |
| 3 | Lecithin | Δ |
| 4 | Polyethylene glycol 400 | ◯ |
| 5 | Simethicon Emulsion 30% | X |
| 6 | Polysorbate 80 | ◯ |
| 7 | Polysorbate 20 | ◯ |
| 8 | a-Tocopherol | Δ |
| 9 | Polyethylene glycol 200 | ◯ |
| 10 | L-Arginine | X |
| 11 | Poloxamer P188 | X |
| 12 | Polyoxyl 40 hydrogenated castor oil | X |
| 13 | Alginic acid | X |

Based on the results in Table 1, primary candidates for CBD solubilizers were selected as those components showing favorable CBD solubilizing properties. As shown in Table 1 below, among the tested components that can be used as solubilizers, corn oil, polyethylene glycol 400, polysorbate 80, polysorbate 20, and polyethylene glycol 200 were considered suitable solubilizers for the CBD aqueous solution. Due to the compatibility required between ingredients for use in the CBD aqueous solution formulation, additional solubilizerswere mixed with the selected primary components, and the extent of CBD dissolution was evaluated.

The symbol “©” indicates a very homogeneous dispersion state; “o” indicates a dispersed state without observable crystals; “A” indicates an unmixed and undispersed state; “X” indicates layer separation, oil clumps, or observable crystals; and “-” indicates that the experiment was not conducted.

TABLE 2

| | | Corn | Polysorbate | Polyethylene | Polyethylene |
|---|---|---|---|---|---|
| No. | Ingredients | oil | 80 | glycol 400 | glycol 200 |
| 1 | Corn oil | — | X | X | X |
| 2 | Glycerin | X | X | X | X |
| 3 | Lecithin | X | X | X | — |
| 4 | Polyethylene | X | Δ | — | — |
| | glycol 400 | | | | |
| 5 | Simethicone | X | X | X | X |
| | Emulsion 30% | | | | |
| 6 | Polysorbate 80 | X | — | Δ | — |
| 7 | Polysorbate 20 | X | Δ | Δ | — |
| 8 | a-Tocopherol | — | X | X | — |
| 10 | Polyethylene | X | X | X | — |
| | glycol 200 | | | | |
| 11 | L-Arginine | X | X | X | X |
| 12 | Poloxamer | X | X | X | X |
| | P407 | | | | |
| 13 | Poloxamer | X | X | X | X |
| | P188 | | | | |
| 14 | Polyoxyl 40 | X | X | X | X |
| | hydrogenated | | | | |
| | castor oil | | | | |
| | (Kolliphor | | | | |
| | RH40) | | | | |
| 15 | Alginic acid | X | X | X | X |

Even components that are generally known as solubilizers were found to be unsuitable for solubilizing CBD isolate. Furthermore, it was confirmed that although some components appeared suitable for mixing with CBD, when combined with other ingredients, the formation of CBD crystals or precipitation was observed, indicating that such combinations were not suitable for preparing a CBD aqueous solution.

### [Preparation of CBD Aqueous Solution Composition]

It was confirmed that the solubility of CBD isolate could be improved by mixing two or more selected special solubilizers. Based on the results of the above experiments, candidates for solubilizers of CBD isolate were selected.

Due to the characteristics of CBD isolate in powder form, issues such as precipitation or aggregation of CBD in water occurred; however, the combinations described below were preliminarily found to provide excellent solubility.

As shown in Table 3 below, the degree of solubility was evaluated using combinations of solubilizers.

TABLE 3

| | Content contained in 1 mL of water (%) | | | |
|---|---|---|---|---|
| | b-9-w | b-9-k | b-14-w | b-14-k |
| | (Preparation | (Preparation | (Preparation | (Preparation |
| | Example 1) | Example 2) | Example 3) | Example 4) |
| CBD | 7.92 | 7.92 | 7.92 | 7.92 |
| Polysorbate 20 | 2.97 | 2.97 | 2.97 | 2.97 |
| Tocopheryl | 1.98 | 1.98 | — | — |
| acetate | | | | |
| Kolliphor RH40 | — | — | 1.98 | 1.98 |
| Poloxamer P188 | — | 7.92 | — | 7.92 |
| Results | Precipitate was | An emulsion | Precipitate | An emulsion |
| (Visual | observed and | was formed. | was observed | was formed. |
| Observation) | no emulsion | No precipitate | and no | An emulsion |
| | was formed. | was visually | emulsion was | was formed. |
| | | observed | formed. | No precipitate |
| | | even after | | was visually |
| | | 2 hours. | | observed |
| | | | | even after |
| | | | | 2 hours. |

Among these combinations, the precipitation rates of CBD in the aqueous solution were evaluated according to CBD concentration using Preparation Examples 2 and 4, which showed particularly excellent solubility. Here, “H” indicates that a high concentration of CBD was added, while “L” indicates that a low concentration of CBD was added.

TABLE 4

| Name | Content (%) | Average Content (%) |
|---|---|---|
| b-9-K-H | 104.63 | 104.64 |
| (high concentration of | 104.58 | |
| Preparation Example 2) | 104.71 | |
| b-9-K-L | 94.11 | 94.02 |
| (low concentration of | 93.93 | |
| Preparation Example 2) | 94.02 | |
| b-14-K-H | 78.40 | 78.36 |
| (high concentration of | 78.37 | |
| Preparation Example 4) | 78.32 | |
| b-14-K-L | 99.48 | 99.47 |
| (low concentration of | 99.41 | |
| Preparation Example 4) | 99.51 | |

The selected Preparation Examples 2 and 4 were analyzed after filtration through a 0.45 μm membrane according to concentration.

Here, the high concentration refers to a CBD isolate content of 110 mg/mL (set concentration), and the low concentration refers to a CBD isolate content of 55 mg/mL (half of the set concentration).

For b-14-k, it was confirmed that the amount of CBD decreased significantly after filtration compared to before filtration. This result indicates that the CBD was not uniformly dispersed and that there was substantial precipitation. It is considered that this was due to reduced solubilization efficiency in water, resulting from decreased saturation solubility and/or microemulsion efficiency.

Based on these results, Preparation Example 2, which showed stable content results at both high and low concentrations, was selected as the final formulation.

### [Final Selected Aqueous Solution Containing CBD Isolate]

TABLE 5

| | Content (%) | |
|---|---|---|
| Ingredients | Example 1 | Comparative Example 1 |
| Cannabidiol (CBD) isolate | 8.16 | 8.16 |
| Polysorbate 20 | 2.04 | 2.04 |
| Tocopheryl acetate | 2.04 | — |
| Tocopherol | — | 2.04 |
| Poloxamer P188 | 4.08 | 4.08 |
| Water | To 100% | To 100% |
| CBD content after 0.45 μm | 104.64 (%) | 29.04% |
| filtration | | |

The aqueous solution composition in which a high content of CBD isolate (purity 99.5%) was dissolved in water corresponds to Example 1 in the above table. In this case, polysorbate 20, tocopheryl acetate, and poloxamer P188 were used as the solubilizers, and no other solubilizer was added. Before manufacturing the orally dissolving film, a CBD aqueous solution was prepared by adding tocopherol instead of tocopheryl acetate and the precipitation rate of CBD after passing through a 0.45 μm filter was evaluated.

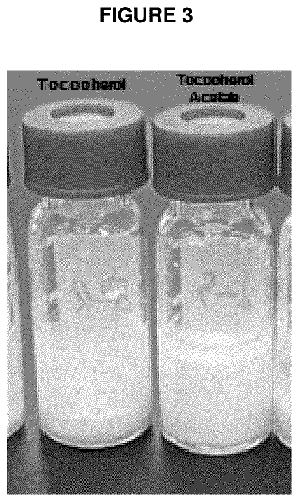

As a result, in Example 1, the CBD content remained unchanged after filtration, whereas in Comparative Example 1, the amount of CBD passing through the filter was greatly reduced. Visual observation also confirmed that the solubilization of CBD in Comparative Example 1 was poorer than in Example 1. These results are shown in FIG. 3.

Conventionally, it has been difficult to dissolve a high concentration of CBD isolate in water, resulting in significant precipitation, but using the composition of the present disclosure allowed stable dissolution of CBD in water without using organic solvents.

Using the CBD aqueous solution, an orally dissolving film containing CBD isolate (Example 2) was prepared with the following composition. Ingredients 1 to 4 were added to water and homogenized using a homogenizer (Ultra Turrax T-25, IKA). Then, the remaining components were added and the mixture homogenized again with the same homogenizer. Subsequently, at 30° C. under vacuum, gas was removed from the film preparation solution, which was then cooled to room temperature, and coated onto a polyethylene terephthalate (PET) film at a suitable thickness. The resulting coated film was dried at 60° C. to obtain an orally disintegrating preparation containing cannabidiol isolate.

TABLE 6
Example 2

| No. | Ingredients | (%) |
|---|---|---|
| 1 | CBD isolate | 7.19 |
| 2 | Polysorbate 20 | 1.8 |
| 3 | Tocopherol acetate | 1.8 |
| 4 | Poloxamer 188 | 3.6 |
| 5 | Polyvinyl alcohol | 8.09 |
| 6 | Xanthan gum | 1.6 |
| 7 | Arabic gum | 1.28 |
| 8 | Other ingredients (Flavor, sweetener, etc.) | 5.58 |
| 9 | water | To 100 |

| | |
|---|---|
| Total (film solution) | 100.00 |

Through the above experiment, an orally dissolving film containing CBD was manufactured. In the film-forming composition, almost no precipitation of CBD isolate was observed. It was confirmed that CBD was stably dispersed and well dissolved in the aqueous solution for film production. Thus, it was found that a film could be easily manufactured using a CBD aqueous solution, rather than an oil-based form.

### [Long-Term Stability of CBD-Containing Film]

The long-term stability of the orally disintegrating film of Example 2, having the composition of Table 6, was evaluated.

For comparison, the long-term stability of CBD in the orally disintegrating film containing CBD-prepared according to the composition shown in Table 7 and corresponding to Korean Patent Application No. 10-2019-0097649—was evaluated as Comparative Example 2.

TABLE 7

| Raw Material | Remarks (%) |
|---|---|
| Hemp powder extract | 30.37 |
| Peppermint oil | 3.64 |
| Sucralose | 4.86 |
| Neohesperidine | 3.04 |
| Tween 80 | 7.65 |
| TiO₂ | 1.37 |
| Xanthan gum | 0.49 |
| HPMC 2910 5cp | 30.37 |
| HPC-L | 18.22 |
| Total | 100% |

Immediately after film formation, the films were packaged in airtight containers and left under harsh conditions for 0, 24, and 72 hours, after which the CBD content was analyzed by high-performance liquid chromatography (HPLC).

The results of evaluating the long-term stability of CBD for CBD isolate, a commercially available CBD formulation, Example 2, and Comparative Example 2 are presented in Table 8. As a result, the orally disintegrating films generally showed a tendency for the CBD content to decrease very rapidly, but the orally disintegrating film of the present invention exhibited excellent retention of CBD content, demonstrating a decline rate similar to that of typical CBD isolate.

TABLE 8

| | | | | | | Decrease | Decrease | |
|---|---|---|---|---|---|---|---|---|
| | Dosage | | | | | (24 | (72 | |
| No. | Form | Sample | 0 hr | 24 hr | 72 hr | hrs, %) | hrs, %) | Remark |
| 0 | Powder | CBD isolate | 103.58 | 103.86 | — | 0.28 | — | CBD isolate |
| | | powder | | | | | | powder (raw) |
| 1 | Liquid | Epidiolex | 100.68 | 59.71 | 53.16 | −40.97 | −47.52 | Reference |
| | | (Reference) | | | | | | product |
| 2 | Film | Comparative | 96.63 | 76.15 | 67.01 | −20.48 | −29.62 | Prior in-house |
| | | Example 2 | | | | | | formulation |
| | | | | | | | | (KR App. No. |
| | | | | | | | | 10-2019- |
| | | | | | | | | 0097649) |
| 3 | Film | Example 2 | 103.63 | 102.29 | 99.24 | −1.34 | −4.39 | Present |
| | | | | | | | | formulation |

Additionally, further experiments were conducted to increase the stability of the orally disintegrating film of the current Example 2. Table 9 below shows the results of evaluating the CBD content over time in films that were prepared by additionally including α-tocopherol and BHT (butylated hydroxytoluene), respectively, in the formulation of the orally disintegrating film of Example 2.

TABLE 9

| | | | | | | Decrease | Decrease | |
|---|---|---|---|---|---|---|---|---|
| | Dosage | | | | | (24 | (72 | |
| No. | Form | Sample | 0 hr | 24 hr | 72 hr | hrs, %) | hrs, %) | Remark |
| 4 | Film | α- | 99.88 | 103.88 | 101.02 | 4.00 | 1.14 | 4 |
| | | tocopherol | | | | | | |
| 5 | Film | BHT | 107.17 | 105.55 | 92.76 | −1.62 | −14.41 | Known |
| | | | | | | | | antioxidant |

As a result, it was confirmed that when α-tocopherol was additionally included, there was no decrease in CBD content. This suggests that α-tocopherol may prevent the decomposition of CBD by blocking contact with water in the microemulsion state, and is presumed to be due to an interaction between α-tocopheryl acetate and α-tocopherol; however, the results described above should not be construed as being limited to such assumptions.

In particular, contrary to expectations that the addition of BHT, a known antioxidant, would prevent CBD decomposition, the addition of BHT actually accelerated CBD degradation, resulting in a rapid decrease in CBD content. When sodium sulfite, a known preservative, was added instead of BHT, a color change occurred immediately after production, making it unsuitable for manufacturing as an orally disintegrating film.

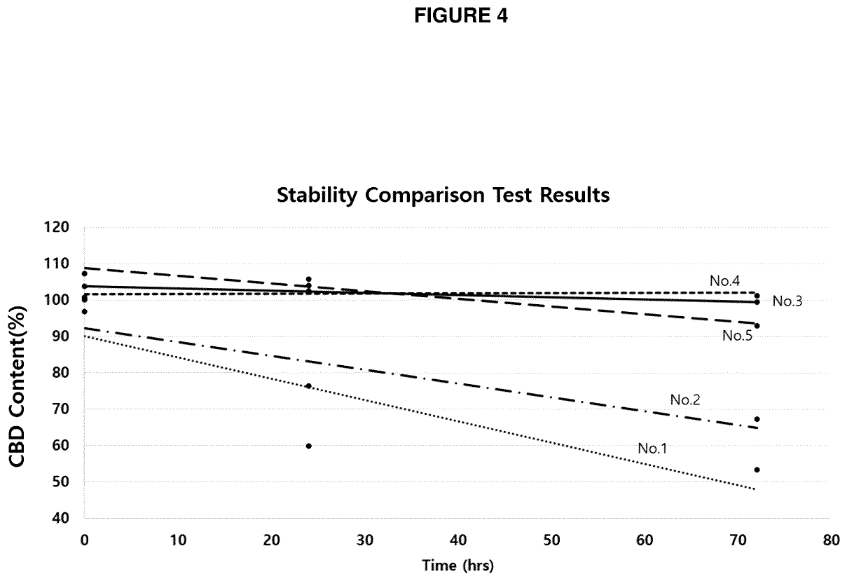

The changes in CBD content described in Tables 8 and 9 above are shown graphically in FIG. 4.

As can be seen in FIG. 4, the formulation of the present invention demonstrates excellent long-term stability. In particular, the formulation provides a CBD-containing film with minimal content variation and addresses the problem of decreasing CBD content, which has been a challenge in CBD-containing formulations.

### INDUSTRIAL APPLICABILITY

The present invention provides a CBD-containing film formulation with improved solubility of cannabidiol.

The present invention also provides a CBD-containing film formulation with enhanced stability of cannabidiol.

1. A cannabidiol aqueous solution composition comprising water as a solvent, wherein the composition comprises:
(a) cannabidiol; and
(b) an additive comprising an ester of tocopherol, polysorbate, and poloxamer.

2. The composition according to claim 1, wherein the (b) additive further comprises alpha-tocopherol (α-tocopherol).

3. The composition according to claim 1, wherein the cannabidiol is highly purified cannabidiol having a purity of 99% or greater.

4. The composition according to claim 1, wherein the (b) additive is present in an amount of 4 to 25% by weight based on the total weight of the aqueous solution composition.

5. The composition according to claim 1, wherein the ester of tocopherol in (b) comprises one or more selected from the group consisting of tocopheryl acetate, tocopheryl linoleate, tocopheryl linoleate/oleate, tocopheryl nicotinate, and tocopheryl succinate.

6. The composition according to claim 1, wherein the polysorbate in (b) comprises one or more selected from the group consisting of polysorbate 20, polysorbate 40, polysorbate 60, and polysorbate 80.

7. The composition according to claim 1, wherein the poloxamer in (b) comprises one or more selected from the group consisting of poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407.

8. The composition according to claim 1, wherein the cannabidiol aqueous solution composition comprises or consists of cannabidiol, tocopheryl acetate, polysorbate 20, poloxamer 188, and water.

9. The composition according to claim 1, wherein the (a) cannabidiol and the (b) additive are mixed in a weight ratio of 1:0.8-1.5.

10. The composition according to claim 1, the additive comprises an ester of tocopherol, polysorbate, and poloxamer, wherein the weight ratio is 1:0.8-1.2:1.8-2.5.

11. The composition according to claim 1, wherein the amount of CBD present in the aqueous solution composition exceeds 100 mg per 1 mL of water, and at least 90% of the CBD in the composition passes through a membrane filter having a pore size of 0.45 μm or less.

12. A cannabidiol oral dissolving film comprising the composition of claim 1 and a water-soluble polymer for film formation.

13. The oral dissolving film according to claim 12, wherein the water-soluble polymer for film formation comprises polyvinyl alcohol and gum.

14. The oral dissolving film according to claim 13, wherein the polyvinyl alcohol has a number-average molecular weight of 15,000 to 60,000.

15. The oral dissolving film according to claim 12, wherein the cannabidiol and water-soluble polymer are mixed in a weight ratio of 1:0.8-1.5.

16. The oral dissolving film according to claim 12, wherein the film comprises cannabidiol in an amount of 15 to 30% by weight based on the total weight of the film.

17. The oral dissolving film according to claim 12, wherein the change in CBD content in the oral dissolving film after storage at a temperature of 60° C. or higher for 72 hours is within ±10% by weight compared to the initial amount at the time of manufacture.

18. The oral dissolving film according to claim 12, wherein the film contains less than 3% by weight of organic solvent(s) other than water based on the total weight of solvents, or does not contain any organic solvent(s) other than water.

19. A method for preparing a cannabidiol purified product aqueous solution comprising:
(S1) preparing a mixture by mixing a cannabidiol purified product with an additive for solubilizing the cannabidiol purified product;
(S2) adding the mixture prepared in step (S1) to a solvent comprising water; and
(S3) stirring the solvent of step (S2),
wherein the cannabidiol purified product contains less than 1% of impurities, and the additive of step (S1) comprises at least one selected from the group consisting of an ester of tocopherol, polysorbate, and poloxamer.

20. The method according to claim 19, wherein the additive further comprises alpha-tocopherol.

21.-23. (canceled)
